(19) United States
(12) Patent Application Publication
(10) Pub. No.: US 2021/0386835 A1
(43) Pub. Date: Dec. 16, 2021

(54) AN ANTIMICROBIAL COMPOSITION FOR SELECTIVELY INHIBITING GROWTH OF P. ACNES BACTERIA

(71) Applicant: CONOPCO, INC., D/B/A UNILEVER, TRUMBULL, CT (US)

(72) Inventors: Sayandip MUKHERJEE, Bangalore (IN); Sandip Bhanudas PATHAK, Bangalore (IN); Anindya DASGUPTA, Bangalore (IN)

(73) Assignee: CONOPCO, INC., D/B/A UNILEVER, TRUMBULL, CT (US)

(21) Appl. No.: 17/287,135

(22) PCT Filed: Nov. 8, 2019
(86) PCT No.: PCT/EP2019/080678

§ 371 Date: Apr. 21, 2021

(30) Foreign Application Priority Data
Nov. 19, 2018 (EP) 18207026.8

(51) Int. Cl. A61K 38/47; A61P 17/10; A61K 31/05; A61K 31/085; A61K 31/045; A61K 9/00; A61P 31/04

(52) U.S. Cl. CPC A61K 38/47; A61P 17/10; A61K 31/05; A61P 31/04; A61K 31/045; A61K 9/0012; A61K 31/085

(57) ABSTRACT

The present invention relates to a method and a composition to prevent or treat acne by selective inhibition of P. acnes bacteria. The method comprises treating skin with P. acnes phage-derived endolysins and nucleic acid molecules encoding the same C in combination with selective essential oils.

### FIELD OF THE INVENTION

The present invention relates to a method and a composition to prevent or treat acne by selective inhibition of P. acnes bacteria. The method comprises treating skin with an endolysin in combination with an essential oil compound.

### BACKGROUND OF THE INVENTION

Acne, also known as Acne vulgaris, is a common skin condition that affects nearly all adolescents and adults at some point in their lives. It has a complex etiology, involving abnormal keratinization, excess sebum production, androgen function, bacterial growth, and immune hypersensitivity. Although one or more of the above processes is correlated with acne, the one triggering factor and the exact sequence of events leading to the formation of acne lesions has not been fully understood. Other factors which have been linked to acne are presence of free radicals with subsequent oxidative stress leading to cellular damage. It has been observed that acne usually occurs in areas rich in sebaceous glands like the face, neck and back.

A bacteria Propionibacterium acnes (P. acnes) has also been implicated in occurrence of acne. It is one of the important and dominant bacteria residing on the human facial skin surface. P. acnes is an aerotolerant anaerobe, slow-growing, rod shaped Gram-positive bacteria. It resides in the sebaceous glands and it constitutes an important part of the skin commensal microbiota. P. acnes uses sebum and by-product from surrounding skin tissue as sources of energy and nutrients. This results in some fatty acid release which can irritate the follicle wall and induce inflammation, leading to acne or acne vulgaris. Acne vulgaris is a chronic, inflammatory disorder of the pilosebaceous gland. It affects almost all adolescents at some point of their lives with 15-20% suffering from moderate to severe forms of acne.

Acne has been treated in many ways. Most treatments take several weeks to months before a noticeable change is seen. Benzoyl peroxide which has an antibacterial effect has been used for mild cases of comedones. In very severe cases of acne, antibiotics like tetracycline, erythromycin and clindamycin have been used. Antibiotics are believed to work by several mechanisms, the most important being the decrease in the number of bacteria in and around the follicle. They are also thought to reduce the irritating chemicals produced by the white blood cells in the sebum, thereby reducing the inflammatory response. However, the drawback of antibiotics and other sort of general antimicrobial treatment is that they are broad-spectrum and help in killing or inhibiting most of the bacteria on skin.

Most of the treatments, as summarized above, involve use of synthetic chemicals. Many people do not prefer use of synthetic chemicals since they are believed to be harsh on the human body. Some people believe that synthetic chemicals cause side effects. Hence, more and more people prefer use of materials which are “natural” e.g. actives based on herbal origin. Further, these days, the actives based on microbiological or biotechnological origin is also coming to be considered as more “natural” then purely synthetic approaches used so far.

Skin, human body's largest organ is colonized by diverse microorganisms. Most of the microorganisms are considered either beneficial or opportunistic potential pathogens. Recent advances in DNA sequencing and computational biology are helping us to identify and understand the beneficial role of these microorganisms on our skin to a greater resolution.

Interactions between skin microorganisms and the human host can be classified into three clusters: 1. Commensalism (one benefits, and no harm occurs to the other), 2. Mutualism (both find benefit), 3. Parasitism (one organisms benefit and other is harmed). Microbes which are not often associated with disease, are mainly known as commensals. Increasing evidence shows that commensal organisms on skin help the immune system to fight against pathogens and maintain homeostasis of the microbiome. One example is Staphylococcus epidermidis (a Gram-positive coagulase negative staphylococci) which plays an important role in acne inhibition. Staphylococcus epidermidis mediates fermentation of glycerol which inhibit overgrowth of P. acnes, leading to acne inhibition. Thus the approach to use broad spectrum antibiotic action which kills or inhibits all the microorganisms on the skin is finding lesser and lesser acceptance among health care practitioners. Increasingly, the approach to selective kill or inhibit microorganism on skin, is finding favour among health care practitioners. Further, the use of antibiotics in treatment of antibiotic resistant strains of P. acnes, is ineffective.

Thus there is a need for finding novel methods of treating problems such as acne in selective fashion. The present inventors thus started working towards providing “natural” solutions to solving the problem of acne. They took up the approach of testing new actives that kill or selectively inhibit growth of P. acnes to the exclusion of other organisms like S. epidermidis, E. coli and S. aureus and experimented with the same. They finally hit up on a combination of P. acnes phage-derived endolysins and an essential oil compound that was found to interact synergistically to selectively inhibit the growth of P. acnes.

US2016346294 (Vyome Biosciences) titled ‘Treatments for resistant acne’ relates generally to novel molecules, compositions, and formulations for treatment of bacterial infections in general and more specifically to bacterial infections with antibiotic resistant pathogens. Exemplified therein is S. capitis (subspecies not specifically mentioned) which is considered a pathogen, not an antimicrobial.

EP2348838B1 (Unilever, 2013) discloses a method of disinfecting surfaces comprising a combination of thymol and terpineol and to a wash off composition comprising the same.

The above two publications do not teach that a combination of P. acnes phage derived endolysin and an essential oil compound can be used to selectively inhibit growth of P. acnes.

It is an object of the present invention to provide for selective kill or inhibition of P. acnes to treat acne.

It is another object of the present invention to provide solution to treating acnes through more natural or nature based actives.

### SUMMARY OF THE INVENTION

According to the first aspect of the present invention there is provided an antimicrobial composition for selectively inhibiting growth of P. acnes bacteria comprising
(i) P. acnes phage-derived endolysins or nucleic acid molecules encoding the same; and
(ii) an essential oil compound of general formula 1 having the structure

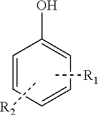

Where R1 is H, OH or OR where R is alkyl chain with 1 to 5 carbon atoms; R2 is a C1 to C6 linear alkyl group; or C3 to C6 branched alkyl group; or C5 to C6 cyclic or heterocyclic alkyl group; or a C6 aromatic group.

According to another aspect of the present invention there is provided a method of controlling or eradicating P. Acnes from skin comprising the step of applying a composition of the invention on to a desired skin surface.

### DETAILED DESCRIPTION OF THE INVENTION

These and other aspects, features and advantages will become apparent to those of ordinary skill in the art from a reading of the following detailed description and the appended claims. For the avoidance of doubt, any feature of one aspect of the present invention may be utilised in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated.

Except in the operating and comparative examples, or where otherwise explicitly indicated, all numbers in this description indicating amounts of material or conditions of reaction, physical properties of materials and/or use are to be understood as modified by the word “about”. Unless specified otherwise, numerical ranges expressed in the format “from x to y” are understood to include x and y. When for a specific feature multiple preferred ranges are described in the format “from x to y”, it is understood that all ranges combining the different endpoints are also contemplated.

The composition as per this invention could be in the form of a leave-on or wash-off format meant for cleaning or disinfecting topical areas e.g. skin and/or hair of mammals, especially humans. Such a composition includes any product applied to a human body for also improving appearance, cleansing, odor control or general aesthetics. The composition of the present invention may be in the form of a liquid, lotion, cream, foam or gel, or toner, or applied with an implement or via a face mask, pad or patch. “Skin” as used herein is meant to include skin on the face and body (e.g., neck, chest, back, arms, underarms, hands, legs, buttocks and scalp). It is especially useful for treatment of acne on the face or any other part of the body where acne forms.

The composition as per the present invention comprises P. acnes phage-derived endolysins and nucleic acid molecules encoding the same. An especially preferred composition comprises P. acnes phage-derived endolysins.

By P. acnes is meant Propionibacterium acnes which is also known as Cutibacterium acnes.

The present inventors looked at various approaches for inhibiting P. Acnes and arrived at the application of bacteriophages and bacteriophage-derived enzymes towards this end. A group of phage-derived enzymes are peptidoglycan (PG) hydrolases known as endolysins. Endolysins are novel muralytic hydrolases encoded by double stranded DNA phages which degrade the PG layer of the bacterial cell wall thereby allowing the progeny phages to escape in the late phase of the infection cycle. Purified endolysins when exposed to PG externally can cause “lysis from outside”. Based on their antimicrobial properties (extraordinary substrate specificity and high activity when added externally) endolysins from phages infecting Gram-positive pathogens has been the motivation and hypothesis for the present inventors as one element of the composition of the present invention.

Thus, it is useful that the endolysin that is included is a recombinant form of P. acnes phage endolysin. This endolysin is preferably cloned from endolysin gene sequence (Gene ID: NC_018851; 855 nucleotide base pair long, 284 amino acids, protein ID: 97935.1) from Propionibacterium phage 29399B_C (GenBank: JX262225.1) which is codon optimized for expression in E. coli and cloned into commercially available pET303/CT-His expression vector.

The nucleotide sequence of endolysin which is especially useful is as per the sequence ID SEQ ID1 which is listed below:

ATGCGTTATATTCCGGCAGCACATCATTCAGCAGGTAGCAATCATCCGGT
TAATCGTGTTGTTATTCATGCAACCTGTCCGGATGTTGGTTTTCCGAGCG
CAAGCCGTAAAGGTCGTGCAGTTAGCACCGCAAACTATTTTGCAAGCCCG
AGCAGCGGTGGTAGCGCACATTATGTTTGTGATATTGGTGAAACCGTTCA
GTGTCTGAGCGAAGGCACCATTGGTTGGCATGCACCGCCTAATCCGCATA
GCCTGGGTATTGAAATTTGTGCAGATGGTGGTAGCCATGCAAGCTTTCGT
GTTCCGGGTCATGCATATACCCGTGAACAGTGGCTGGATCCGCGTGTTTG
GCCTGCAGTTGAAAAAGCAGCAATTCTGTGTCGTCGTCTGTGCGATAAAT
ACAATGTTCCGAAACGTAAACTGAGCGCAGCAGATCTGAAAGCAGGTCGT
CGTGGTGTTTGTGGTCATGTTGATGTTACCGATGCATGGCATCAGAGCGA
TCATGATGATCCGGGTCCGTGGTTTCCGTGGGATCGTTTTATGGCAGTTG
TTAATGGTCATAACGAAAGCGGTGAACTGACCGTTGCAGATGTTAAAGCA
CTGCATGATCAGATTAAACAGCTGAGCGCACAGCTGGCAGGTAGCGTTAA
TAAACTGCATCATGATGTTGGTGTTGTTCAGGTTCAGAATGGTGATCTGG
GTAAACGTGTTGATGCACTGAGCTGGGTTAAAAATCCGGTTACCGGTAAA
CTGTGGCGTACCAAAGATGCACTGTGGTCAGTTTGGTATTATGTTCTGGA
ATGTCGTAGCCGTATTGATCGTCTGGAAAGCGCAGTTAATGGTCTGAAAA
AACTCGAGCACCACCACCACCACCACTGAGATCCGGCTGCTAA.

An especially preferred aspect relates to the endolysin where the stop codon was removed from the 3′ end of the gene to accommodate a 6× Histidine tag.

Polypeptide sequence of Endolysin with C-terminal Histidine tag (×6)

MRYIPAAHHSAGSNHPVNRVVIHATCPDVGFPSASRKGRAVSTANYFASP
SSGGSAHYVCDIGETVQCLSEGTIGWHAPPNPHSLGIEICADGGSHASFR
VPGHAYTREQWLDPRVWPAVEKAAILCRRLCDKYNVPKRKLSAADLKAGR
RGVCGHVDVTDAWHQSDHDDPGPWFPWDRFMAVVNGHNESGELTVADVKA
LHDQIKQLSAQLAGSVNKLHHDVGVVQVQNGDLGKRVDALSWVKNPVTGK
LWRTKDALWSVWYYVLECRSRIDRLESAVNGLKKLEHHHHHH

It is also possible that the nucleic acid molecules of the endolysin for optional inclusion in the composition of the invention comprise fragments, variants and fusions of the endolysin which are capable of specifically binding to and/or lysing cells of P. acnes.

The composition of the invention includes an essential oil compound of general formula having the structure

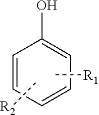

Where R1 is H, OH or OR where R is an alkyl chain with 1 to 5 carbon atoms; R2 is a C1 to C6 linear alkyl group; or C3 to C6 branched alkyl group; or C5 to C6 cyclic or heterocyclic alkyl group; or a C6 aromatic group.

The most preferred essential oil compounds as per compound of formula 2 for use in the composition of the invention are selected from thymol, carvacrol, (E)-2(prop-1-enyl) phenol, 2-propylphenol, 4-pentylphenol, 4-sec-butylphenol, 2-benzyl phenol, or eugenol or combinations thereof.

The structure of these compounds are given below:

The structure of thymol is given below:

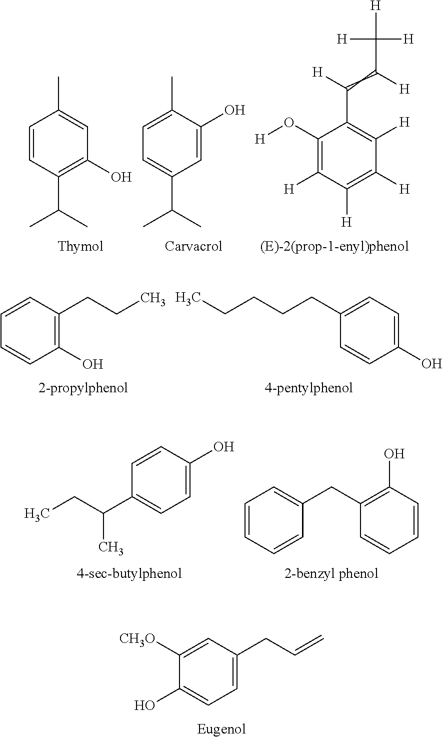

Of the above essential oil compounds, thymol, carvacrol, 4-pentyl phenol, or 4-sec-butylphenol. are more preferred for use in the composition of the invention. The most preferred essential oil compound is thymol.

The essential oil compounds are preferably included in 0.001 to 1%, preferably 0.005 to 1%, further more preferably 0.005 to 0.5% by weight of the composition.

It is further more preferred that an additional essential oil compound of the terpene group is included in the composition of the invention. The most preferred compound of the terpene group is terpineol. The terpineol is preferably selected from alpha-terpineol, beta-terpineol, gamma-terpineol or mixtures thereof. It is particularly preferred that the terpineol is alpha-terpineol. Terpineol may be added to the antimicrobial composition in purified form.

The structure of a terpineol compound is given below:

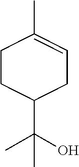

The antimicrobial composition preferably comprises 0.01 to 1% of terpineol more preferably from 0.05 to 0.5%, by weight of the composition.

As per an especially preferred aspect of the invention, the composition comprises a mixture of thymol and terpineol.

Without wishing to be bound by theory, the present inventors believe that in combination, the essential oils and endolysin synergistically target two different substrates critical to the bacterial defence. They believe that for Gram-positive bacteria like P. acnes, endolysins perturb the outermost coat of the bacteria (i.e. cell wall) providing rapid access for thymol to target the lipid fractions in the inner cell membrane of the bacteria. Together this accounts for rapid and specific lysis of P. acnes.

The composition of the invention preferably includes a topically acceptable carrier. Preferred topically acceptable carrier may comprise an anhydrous base, a gel, a lotion, a cream or an emulsion.

The composition of the invention is preferably a wash-off composition and this is enabled by including 1 to 80% by weight of a surfactant. In general, the surfactants may be chosen from the surfactants described in well known textbooks like “Surface Active Agents” Vol. 1, by Schwartz & Perry, Interscience 1949, Vol. 2 by Schwartz, Perry & Berch, Interscience 1958, and/or the current edition of “McCutcheon's Emulsifiers and Detergents” published by Manufacturing Confectioners Company or in “Tenside-Taschenbuch”, H. Stache, 2nd Edn., Carl Hauser Verlag, 1981. Any type of surfactant, i.e. anionic, cationic, nonionic, zwitterionic or amphoteric can be used.

A particularly preferred surfactant is soap. Soap is a suitable surfactant for personal washing applications of composition of the invention. The soap is preferably C8-C24 soap, more preferably C10-C20 soap and most preferably C12-C16 soap. The soap may or may not have one or more carbon-carbon double bond or triple bond. The cation of the soap may be alkali metal, alkaline earth metal or ammonium. Preferably, the cation of the soap is selected from sodium, potassium or ammonium. More preferably the cation of the soap is sodium or potassium.

The soap may be obtained by saponifying a fat and/or a fatty acid. The fats or oils generally used in soap manufacture may be such as tallow, tallow stearines, palm oil, palm stearines, soya bean oil, fish oil, castor oil, rice bran oil, sunflower oil, coconut oil, babassu oil, palm kernel oil, and others. In the above process the fatty acids are derived from oils/fats selected from coconut, rice bran, groundnut, tallow, palm, palm kernel, cotton seed, soyabean, castor etc.

A typical fatty acid blend consisted of 5 to 30% coconut fatty acids and 70 to 95% fatty acids ex hardened rice bran oil. Fatty acids derived from other suitable oils/fats such as groundnut, soybean, tallow, palm, palm kernel, etc. may also be used in other desired proportions. The most preferred soap is a laurate soap. The soap, when present in solid forms of the present invention is present in an amount of 30 to 90%, preferably from 50 to 85%, more preferably 55 to 75% by weight of the composition. The soap, when present in liquid forms of the composition is present in 0.5 to 20%, preferably from 1 to 10% by weight of the composition.

Alternately the surfactants are non-ionic surfactants, such as C8-C22, preferably C8-C16 fatty alcohol ethoxylates, comprising between 1 and 8 ethylene oxide the surfactants are preferably selected from primary alkyl sulphate, secondary alkyl sulphonates, alkyl benzene sulphonates, or ethoxylated alkyl sulphates. The composition may further comprise an anionic surfactant, such as alkyl ether sulphate preferably those having between 1 and 3 ethylene oxide groups, either from natural or synthetic source and/or sulphonic acid. Especially preferred are sodium lauryl ether sulphates. Alkyl polyglucoside may also be present in the composition, preferably those having a carbon chain length between C6 and C16. Suitable surfactant concentrations in liquid forms of cleaning application are generally more than 0.5 but less than 10%, preferably from 1 to 5% by weight of the composition. In solid compositions, the surfactant is preferably present in 5 to 40%, preferably from 10 to 30% by weight of the composition. Water may preferably be present in 10 to 90% by weight of the composition depending on the format of the composition. In solid composition water may be present in 10-30%, while in liquid or semi-solid composition, water may be present in 40 to 90%.

The composition of the invention is especially suitable for use in a wash off process where the contact time of the antimicrobial actives with the surface is low, i.e of the order of less than 5 minutes, preferably less than 2 minutes, further more preferably less than a minute and in many cases less than 15 seconds.

When the composition in accordance with the invention is a leave on composition, it preferably comprises one or more of fragrance, surfactant, organic sunscreen, inorganic sunscreen, emollient, humectant, extender pigment and preservative.

Sunscreens include those materials which block harmful ultraviolet light. Preferred suncreens are the derivatives of p-aminobenzoic acid (PABA), cinnamate and salicylate. For example, avobenzophenone (Parsol® 1789), octyl methoxycinnamate and 2-hydroxy-4-methoxy benzophenone (also known as oxybenzone) can be used. Octyl methoxycinnamate and 2-hydroxy-4-methoxy benzophenone are commercially available under the trade marks, Parsol® MCX and Benzophenone-3, respectively. Ecamsule®, a benzylidene camphor derivative, and drometrizole trisiloxane, a benzotriazole, may also be used. Further examples include Octocrylene, phenylbenzimidazole sulfonic acid (also known as Ensulizole), ethylhexyl salicylate, diethylhexyl naphthylate, bimotrizinole (trade marked as Tinosorb® S) and bisoctrizole (Tinosorb® M). Inorganic sunscreens include oxides like titanium dioxide and zinc oxide which reflect or scatter the sunrays. The quantity of sunscreens present in the compositions could vary depending upon the degree of protection desired from UV radiation. Preferably, the compositions comprise 0.01 to 15% by weight, more preferably 0.1 to 10 and most preferably 0.5 to 7.5% by weight sunscreen.

Illustrative examples of the types of fragrances that may be used include those comprising terpenes and terpene derivatives like those described in Bauer, K., et al., Common Fragrance and Flavor Materials, VCH Publishers (1990). Further examples include myrcene, dihydromyrenol, citral, tagetone, cis-geranic acid, citronellic acid, mixtures thereof.

The carrier acts as diluent or dispersant for the ingredients of the compositions. The carrier may be aqueous-based, anhydrous or an emulsion, whereby a water-in-oil or oil-in-water emulsion is generally preferred. If the use of water is desired, water typically makes up the balance of the composition, which most preferably is from 40 to 80% by weight of the composition.

In addition to water, organic solvents may optionally be included as carrier to assist any other carrier in the compositions of the present invention. Examples include alkanols like ethyl and isopropyl alcohol.

Other suitable organic solvents include ester oils like isopropyl myristate, cetyl myristate, 2-octyldodecyl myristate, avocado oil, almond oil, olive oil and neopentylglycol dicaprate. Typically, such ester oils assist in emulsifying the compositions, and an effective amount is often used to yield a stable, and most preferably, water-in-oil emulsion.

Emollients may also be used, if desired, as a carrier. Alcohols like 1-hexadecanol (i.e. cetyl alcohol) are preferred. Other emollients include silicone oils and synthetic esters. Silicone oils suitable for use include cyclic or linear polydimethylsiloxanes containing from 3 to 9, preferably from 4 to 5 silicon atoms. Non-volatile silicone oils useful as emollients include polyalkyl siloxanes, polyalkylaryl siloxanes and polyether siloxane copolymers. The non-volatile polyalkyl siloxanes useful polydimethylsiloxanes. Silicone elastomers may also be used. The ester emollients that may optionally be used are:
(i) alkenyl or alkyl esters of fatty acids having 10 to 20 carbon atoms. Examples thereof include isoarachidyl neopentanoate, isononyl isonanonoate, oleyl myristate, oleyl stearate, and oleyl oleate;
(ii) ether-esters such as fatty acid esters of ethoxylated fatty alcohols;
(iii) polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters;
(iv) wax esters such as beeswax, spermaceti, stearyl stearate and arachidyl behenate; and,
(v) sterols esters, of which cholesterol fatty acid esters are examples.

Emollients, when present, typically make up from 0.1 to 50% by weight of the composition, including all ranges subsumed therein.

Fatty acids having from 10 to 30 carbon atoms may also be included as carriers. Examples of such fatty acids include pelargonic, lauric, myristic, palmitic, stearic, isostearic, oleic, linoleic, arachidic, behenic or erucic acid and mixtures thereof.

Humectants of the polyhydric alcohol type may also be employed in the compositions. The humectant often aids in increasing the effectiveness of the emollient, reduces scaling at the skin surface, stimulates removal of built-up scale and improves skin feel. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results, the humectant is preferably propylene glycol or sodium hyaluronate. Other humectants which may be used include hydroxyethyl urea. The amount of humectant may be 0.2 to 25% by weight and preferably from 0.5 to 15% by weight of the composition.

Moisturisation may be improved through use of petrolatum or paraffin. Thickeners may also be utilized as a portion of the carrier in the compositions. Typical thickeners include cross-linked acrylates (e.g. Carbopol® 982), hydrophobically-modified acrylates (e.g. Carbopol® 1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Amounts of the thickener may range from 0.001 to 5, optimally from 0.01 to 0.5% by weight of the composition.

Surfactants may also be present. When present, the total amount of surfactants is 2 to 40% by weight, and preferably from 4 to 20% by weight, optimally from 5 to 12% by weight of the composition. The surfactant is selected from the group consisting of anionic, nonionic, cationic and amphoteric actives. Particularly preferred nonionic surfactants are those with a 010-20 fatty alcohol or acid hydrophobe condensed with from 2 to 100 moles of ethylene oxide or propylene oxide per mole of hydrophobe; mono- and di-fatty acid esters of ethylene glycol; fatty acid monoglyceride; sorbitan, mono- and di-C8-C20 fatty acids; block copolymers (ethylene oxide/propylene oxide); and polyoxyethylene sorbitan as well as combinations thereof. Alkyl polyglycosides and saccharide fatty amides (e.g. methyl gluconamides) are also suitable nonionic surfactants.

Preferred anionic surfactants include soap, alkyl ether sulfate and sulfonates, alkyl sulfates and sulfonates, alkylbenzene sulfonates, alkyl and dialkyl sulfosuccinates, C_{8 to 20} acyl isethionates, acyl glutamates, C_{8 to 20} alkyl ether phosphates and combinations thereof.

Various other ingredients may also be used in compositions. Actives are defined as skin benefit agents other than emollients and other than ingredients that merely improve the physical characteristics of the composition. Although not limited to this category, general examples include extender pigments such as talcs and silicas, as well as alpha-hydroxy acids, beta-hydroxy acids and zinc salts.

Beta-hydroxy acids include salicylic acid. Zinc oxide and zinc pyrithione are examples of useful zinc salts.

Compositions, especially those containing water, need to be protected against harmful microorganisms. Anti-microbial compounds, such as triclosan, and preservatives may become necessary. Suitable preservatives include alkyl esters of p-hydroxybenzoic acid, hydantoin derivatives, propionate salts, and a variety of quaternary ammonium compounds. Particularly preferred preservatives are methyl paraben, propyl paraben, phenoxyethanol and benzyl alcohol. Preservatives are from 0.1 to 2% by weight of the composition.

The packaging could be a patch, bottle, tube, roll-ball applicator, propellant driven aerosol device, squeeze container or lidded jar.

According to another aspect of the present invention there is provided a method of controlling or eradicating P. acnes from skin comprising the step of applying a composition of the invention on to a desired skin surface. The method is preferably non-therapeutic. The method is especially useful for reducing or eliminating acne on skin.

The invention will now be illustrated with the help of the following non-limiting examples.

### EXAMPLES

Synergistic effect of combination of Propionibacterium acnes phage-derived recombinant endolysin and thymol; and thymol+terpineol.

P. acnes 6919 strain (ATCC, USA) was grown under anaerobic conditions on RCM agar from Glycerol stocks. A loopful of culture from glycerol stock was streaked on freshly prepared RCM agar plate and incubated at 37° C. anaerobically for 4-5 days. After the incubation, the culture was taken from the plate and re-suspended in saline and then centrifuged at 8000 rpm for 6 minutes at 4° C. The pellet obtained was re-suspended in 10 mM HEPES buffer (pH 7.0). An OD₆₀₀ culture of 0.5 was made in HEPES buffer. 1:10 dilution of 0.5 OD adjusted culture was used for contact kill assay corresponding to 10⁶ cells per ml.

The assay was done in 200 μl total volume. 14 μl of purified endolysin (final conc. 100 μg/ml) was used for the assay. 10% w/v of essential oil was prepared fresh in absolute ethanol. The contact assay was performed in 10 mM HEPES buffer (pH 7.0). For control, 181 μl of 1:10 diluted 0.6 OD₆₀₀ adjusted culture was mixed with 19 μl of HEPES buffer to make a volume of 200 μl. Similarly, For Endolysin and essential oil, required amounts of protein were mixed with P. acnes culture for individual & for combination treatment. The mixture was mixed and incubated at 37° C. for 2 hours in shaker incubator.

Post incubation, the samples were neutralised by serial 1:10 dilution in D/E broth followed by saline dilutions. 100 μl of each dilution was plated on fresh RCM agar plates and allowed to dry. The plates were sealed with parafilm and incubated at 37° C. in anaerobic chamber for 4-5 days. Post incubation, colonies were counted and titer was estimated as log CFU/ml. The relative log reductions of individual and combination treatment were calculated with respect to control and the data is presented in the table below:

| | Relative log |
|---|---|
| Sample | reduction |
| Endolysin (3 μM) | 1.17 |
| Thymol (0.01%) | 0.33 |
| Endolysin (3 μM) + Thymol (0.01%) | 2.72 |
| Thymol (0.02%) | 2.02 |
| Endolysin (3 μM) + Thymol (0.02%) | 3.28 |
| Thymol (0.015%) + Terpineol (0.015%) | 1.37 |
| Endolysin (3 μM) + Thymol (0.015%) + | 3.42 |
| Terpineol (0.015%) | |
| Thymol (0.025%) + Terpineol (0.025%) | 2.48 |
| Endolysin (3 μM) + Thymol (0.025%) + | 3.48 |
| Terpineol (0.025%) | |

The data in the table above shows synergistic antimicrobial activity of endolysin and thymol and also for its combination with thymol and terpineol against P. acnes for a combined exposure of 2 hours, over various concentration ranges.

1. An antimicrobial composition for selectively inhibiting growth of Propionibacterium acnes bacteria comprising
(i) P. acnes phage-derived endolysins or nucleic acid molecules encoding the same; and
(ii) an essential oil compound of general formula 1 having the structure 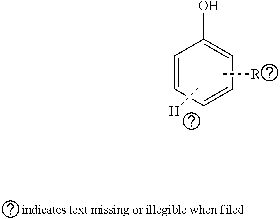
Where R₁ is H, OH or OR, where R is an alkyl chain with 1 to 5 carbon atoms; R₂ is a C₁ to C₆ linear alkyl group; C₃ to C₆ branched alkyl group; C₅ to C₆ cyclic or heterocyclic alkyl group; or a C₆ aromatic group.

2. The antimicrobial composition as claimed in claim 1, wherein said endolysin is a recombinant form of P. acnes phage endolysin.

3. The antimicrobial composition as claimed in claim 1, wherein said endolysin is cloned from endolysin gene sequence (Gene ID: NC_018851; 855 nucleotide base pair long, 284 amino acids, protein ID: 97935.1) from Propionibacterium phage 29399B_C (GenBank: JX262225.1) which is codon optimized for expression in E. coli and cloned into commercially available pET303/CT-His expression vector.

4. The antimicrobial composition as claimed in claim 1, wherein the stop codon was removed from the 3′ end of the gene to accommodate a 6× Histidine tag.

5. The antimicrobial composition as claimed in claim 1, wherein nucleotide sequence of endolysin is SEQ ID1:
ATGCGTTATATTCCGGCAGCACATCATTCAGCAGGTAGCAATCATCCGGT
TAATCGTGTTGTTATTCATGCAACCTGTCCGGATGTTGGTTTTCCGAGCG
CAAGCCGTAAAGGTCGTGCAGTTAGCACCGCAAACTATTTTGCAAGCCCG
AGCAGCGGTGGTAGCGCACATTATGTTTGTGATATTGGTGAAACCGTTCA
GTGTCTGAGCGAAGGCACCATTGGTTGGCATGCACCGCCTAATCCGCATA
GCCTGGGTATTGAAATTTGTGCAGATGGTGGTAGCCATGCAAGCTTTCGT
GTTCCGGGTCATGCATATACCCGTGAACAGTGGCTGGATCCGCGTGTTTG
GCCTGCAGTTGAAAAAGCAGCAATTCTGTGTCGTCGTCTGTGCGATAAAT
ACAATGTTCCGAAACGTAAACTGAGCGCAGCAGATCTGAAAGCAGGTCGT
CGTGGTGTTTGTGGTCATGTTGATGTTACCGATGCATGGCATCAGAGCGA
TCATGATGATCCGGGTCCGTGGTTTCCGTGGGATCGTTTTATGGCAGTTG
TTAATGGTCATAACGAAAGCGGTGAACTGACCGTTGCAGATGTTAAAGCA
CTGCATGATCAGATTAAACAGCTGAGCGCACAGCTGGCAGGTAGCGTTAA
TAAACTGCATCATGATGTTGGTGTTGTTCAGGTTCAGAATGGTGATCTGG
GTAAACGTGTTGATGCACTGAGCTGGGTTAAAAATCCGGTTACCGGTAAA
CTGTGGCGTACCAAAGATGCACTGTGGTCAGTTTGGTATTATGTTCTGGA
ATGTCGTAGCCGTATTGATCGTCTGGAAAGCGCAGTTAATGGTCTGAAAA
AACTCGAGCACCACCACCACCACCACTGAGATCCGGCTGCTAA.

6. The antimicrobial composition as claimed in claim 1, wherein the nucleic acid molecules comprise fragments, variants and fusions of the endolysin which are capable of specifically binding to and/or lysing cells of P. acnes.

7. The antimicrobial composition as claimed in claim 1, wherein said essential oil compound comprises thymol, carvacrol, (E)-2(prop-1-enyl) phenol, 2-propyl phenol, 4-pentyl phenol, 4-sec-butylphenol, 2-benzyl phenol, eugenol or combinations thereof.

8. The antimicrobial composition as claimed in claim 7, wherein the essential oil compound is thymol, carvacrol, 4-pentyl phenol, or 4-sec-butylphenol.

9. The antimicrobial composition as claimed in claim 1, wherein the antimicrobial composition additionally comprises terpineol.

10. The antimicrobial composition as claimed in claim 9 comprising a mixture of terpineol and the essential oil compound thymol.

11. The antimicrobial composition as claimed in claim 1 additionally comprising a topically acceptable carrier.

12. The antimicrobial composition as claimed in claim 11, wherein the topically acceptable carrier comprises an anhydrous base, a gel, lotion, cream or emulsion.

13. A method of controlling or eradicating P. acnes from skin comprising the step of applying an antimicrobial composition on to a desired skin surface as claimed in claim 1.

14. The antimicrobial composition as claimed in claim 7, wherein the essential oil compound is thymol.

15. The antimicrobial composition as claimed in claim 1, wherein the antimicrobial composition comprises 0.0001 to 1% of essential oil compound by weight of the antimicrobial composition.

16. The antimicrobial composition as claimed in claim 9, wherein the antimicrobial composition comprises 0.01 to 1% of terpineol by weight of the antimicrobial composition.

17. The antimicrobial composition as claimed in claim 9, wherein the terpineol comprises alpha-terpineol, beta-terpineol, gamma-terpineol or mixtures thereof.

18. The antimicrobial composition as claimed in claim 1, wherein the antimicrobial composition is a wash-off composition further comprising surfactant.

19. The antimicrobial composition as claimed in claim 18, wherein the antimicrobial composition comprises 1 to 80% of surfactant by weight of the antimicrobial composition.


SEQUENCE LISTING (2 sequences)

SEQ ID NO: 1 - DNA, 893 bp, Artificial Sequence
  Nucleotide sequence of endolysin
atgcgttatattccggcagcacatcattcagcaggtagcaatcatccggttaatcgtgttgttattcatgcaacctgtccggatgttggttttccgagcgcaagccgtaaaggtcgtgcagttagcaccgcaaactattttgcaagcccgagcagcggtggtagcgcacattatgtttgtgatattggtgaaaccgttcagtgtctgagcgaaggcaccattggttggcatgcaccgcctaatccgcatagcctgggtattgaaatttgtgcagatggtggtagccatgcaagctttcgtgttccgggtcatgcatatacccgtgaacagtggctggatccgcgtgtttggcctgcagttgaaaaagcagcaattctgtgtcgtcgtctgtgcgataaatacaatgttccgaaacgtaaactgagcgcagcagatctgaaagcaggtcgtcgtggtgtttgtggtcatgttgatgttaccgatgcatggcatcagagcgatcatgatgatccgggtccgtggtttccgtgggatcgttttatggcagttgttaatggtcataacgaaagcggtgaactgaccgttgcagatgttaaagcactgcatgatcagattaaacagctgagcgcacagctggcaggtagcgttaataaactgcatcatgatgttggtgttgttcaggttcagaatggtgatctgggtaaacgtgttgatgcactgagctgggttaaaaatccggttaccggtaaactgtggcgtaccaaagatgcactgtggtcagtttggtattatgttctggaatgtcgtagccgtattgatcgtctggaaagcgcagttaatggtctgaaaaaactcgagcaccaccaccaccaccactgagatccggctgctaa

SEQ ID NO: 2 - protein, 292 aa, Artificial Sequence
  Polypeptide sequence of Endolysin with C-terminal Histidine tag (x6)
MRYIPAAHHSAGSNHPVNRVVIHATCPDVGFPSASRKGRAVSTANYFASPSSGGSAHYVCDIGETVQCLSEGTIGWHAPPNPHSLGIEICADGGSHASFRVPGHAYTREQWLDPRVWPAVEKAAILCRRLCDKYNVPKRKLSAADLKAGRRGVCGHVDVTDAWHQSDHDDPGPWFPWDRFMAVVNGHNESGELTVADVKALHDQIKQLSAQLAGSVNKLHHDVGVVQVQNGDLGKRVDALSWVKNPVTGKLWRTKDALWSVWYYVLECRSRIDRLESAVNGLKKLEHHHHHH